(19) United States
(12) Patent Application Publication
(10) Pub. No.: US 2013/0110008 A1
(43) Pub. Date: May 2, 2013

(54) COMMUNICATION BETWEEN EXTERNAL DEVICES AND IMPLANTABLE MEDICAL DEVICES

(71) Applicants: Duane L. Bourget, Albertville, MN (US); Jay T. Eisch, Wyoming, MN (US); Yu Wang, Plymouth, MN (US)

(72) Inventors: Duane L. Bourget, Albertville, MN (US); Jay T. Eisch, Wyoming, MN (US); Yu Wang, Plymouth, MN (US)

(73) Assignee: MEDTRONIC, INC., Minneapolis, MN (US)

(21) Appl. No.: 13/284,696

(22) Filed: Oct. 28, 2011

(51) Int. Cl. A61B 5/11; H04W 12/04; H04W 12/06; A61B 5/103

(52) U.S. Cl. 600/595; 600/587; 380/270

(57) ABSTRACT

In general, the techniques of this disclosure are directed to communication between an implantable medical device (IMD) and an external device. In some examples, the external device transmits a signal that includes a communication key. One or more sensors of the IMD sense the signal that includes the communication key, and the IMD uses the communication key for coding the communication between the IMD and the external device. The one or more sensors that sensed the signal may also sense one or more patient characteristics.

### TECHNICAL FIELD

This disclosure relates to implantable medical devices (IMDs), and, more particularly, to communication with IMDs.

### BACKGROUND

An implantable medical device (IMD) and an external device such as a programmer wirelessly communicate with one another. For patient privacy reasons and to ensure that the IMD is communicating with the proper programmer, the wireless communication is generally coded, e.g., encrypted. For example, the programmer wirelessly transmits a communication key to the IMD. The IMD uses the communication key to decode additional information transmitted by the programmer. The IMD may also use the communication key to encode information that it transmits to the programmer.

### SUMMARY

In general, the techniques described in this disclosure are directed to utilizing, for communication purposes, one or more sensors within an implantable medical device (IMD) that are also used for sensing patient characteristics. For instance, a sensor within the IMD that senses patient characteristics may also sense a signal that includes a communication key. The IMD may utilize the communication key that is included in the signal sensed by the sensor to decode received information and/or encode transmitted information.

In one example, the disclosure describes a method that includes sensing, with a sensor of an implantable medical device (IMD), a first signal that includes a communication key, and receiving, with the IMD, the communication key from the sensed first signal. The method also includes coding, with the IMD, at least one of a second signal transmitted by the IMD and a second signal received by the IMD with the communication key received from the sensor, and sensing, with the sensor that sensed the first signal, a patient characteristic.

In another example, the disclosure describes an implantable medical device (IMD) that includes a sensor, a telemetry module, and a processor. The sensor is operable to sense a first signal that includes a communication key and is operable to sense a patient characteristic. The processor is operable to receive the communication key from the sensed first signal, and code at least one of a second signal transmitted by the telemetry module and a second signal received by the telemetry module with the communication key received from the sensor.

In another example, the disclosure describes a system that includes at least one external device that transmits a first signal that includes a communication key, at least one lead that includes a sensor operable to sense the first signal that includes the communication key and operable to sense a patient characteristic, and an implantable medical device (IMD) implanted within a patient and coupled to the at least one lead. In this example, the IMD includes a telemetry module, and a processor operable to receive the communication key from the sensed first signal, and code at least one of a second signal transmitted by the telemetry module and a second signal received by the telemetry module with the communication key received from the sensor.

In another example, the disclosure describes an implantable medical device (IMD) that includes means for sensing a first signal that includes a communication key and a patient characteristic. The IMD also includes means for receiving the communication key from the sensed first signal, and means for coding at least one of a second signal transmitted by the IMD and a second signal received by the IMD with the communication key received from the means for sensing.

In another example, the disclosure describes a non-transitory computer-readable storage medium comprising instructions that, in response to a sensor of an implantable medical device (IMD), which is operable to sense a patient characteristic, sensing a first signal that includes a communication key, cause one or more processors to receive the communication key from the sensed first signal. The instructions also causes the one or more processors to code at least one of a second signal transmitted by the IMD and a second signal received by the IMD with the communication key received from the sensor.

The details of one or more aspects of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a block diagram illustrating an implantable medical device (IMD) of a system that includes the IMD and an external device.

FIG. 2 is a block diagram illustrating an IMD of a system that includes the IMD and two external devices.

FIGS. 3A and 3B are timing diagrams illustrating examples of acoustic signals.

FIG. 4 is a flowchart illustrating an example operation of an IMD.

FIG. 5 is a block diagram illustrating an example external device.

FIG. 6 is a schematic diagram illustrating an implantable medical device (IMD) implanted within a patient.

### DETAILED DESCRIPTION

An implantable medical device (IMD) and an external device such as a programmer may communicate information to one another using a secure telemetry system. Communication between the IMD and the external device may include an initialization phase and a transfer phase. In the initialization phase, the external device may transmit a wake-up command and an authentication key. During the initialization phase, the external device may also transmit a communication key to the IMD. The IMD may utilize the communication key to decrypt information received in the transfer phase, or encrypt the information that the IMD transmits in the transfer phase. In this manner, the IMD and the external device may support secure communication using encryption. In some examples, the external device may transmit the wake-up command, the authentication key, and the communication key as three separate signals. In other examples, the external device may transmit a combination of the wake-up command, the authentication key, and the communication key in a single signal or in two separate signals.

During the transfer phase, the IMD may receive information from the external device, and/or transmit information to the external device. Alternatively, during the transfer phase, the IMD may receive information from a device other than the external device that transmitted the wake-up command, authentication key, and communication key. For purposes of illustration, one or more of the examples are described in the context where the external device transmits information during the initialization phase and transmits and receives information during the transfer phase. However, aspects of this disclosure are not so limited.

Because information received by the IMD may be personal and sensitive information, the external device may encrypt the information that it transmits to the IMD to support secure communication. For example, the external device may encrypt the information in such a manner that the information can only be decrypted with the communication key that the IMD received during the initialization phase. The IMD may then utilize its received communication key to decrypt the encrypted information. In the reverse, for patient privacy, during the transfer phase, the IMD may encrypt information that the IMD transmits to the external device such that the encrypted information can only be decrypted with the communication key. The external device may decode the received information using the communication key.

In this way, devices other than the IMD and the external device may not be able to decipher information communicated between the IMD and the external device, thereby protecting patient privacy. For example, if there are multiple external devices in the vicinity of the IMD, information transmitted to the intended external device by the IMD may not be decipherable by any other external device other than the intended external device. Also, in this way, information transmitted by the external device may not cause another IMD, other than the intended IMD, to be inadvertently programmed to perform unintended functions. For instance, during the transfer phase, some other IMD may be in the vicinity of the external device, and may sense the signal transmitted by the external device to the intended IMD. Because this other IMD may not have the communication key, it may not be able to decrypt the transmitted information, whereas the intended IMD would be able to decrypt the transmitted information with the communication key, and perform functions instructed by the information.

In some examples, the IMD and the external device may utilize different communication techniques during the initialization phase and the transfer phase. For example, the signal or signals that the IMD receives during the initialization phase may be acoustic signals. In this example, the IMD may receive one or more acoustic signals that include the communication key, the wake-up command, and the authentication key.

The signal transmitted or received by the IMD and the external device during the transfer phase may not be an acoustic signal. For example, during the transfer phase, the external device may transmit a radio frequency (RF) signal, and the IMD may receive the RF signal. Similarly, during the transfer phase, the IMD may transmit an RF signal, and the external device may receive the RF signal. In this example, the digital bits of the RF signal may be encoded and decoded with the communication key.

In some alternate examples, it may be possible for the IMD to receive the wake-up command and the authentication key as RF signals. For example, the external device may transmit the wake-up command and the authentication key as one or more RF signals. In these alternate examples, the IMD may still receive the communication key as an acoustic signal. Also, in these alternate examples, the IMD and the external device may communicate with one another, during the transfer phase, using RF signals. For purposes of brevity, the example techniques described in this disclosure are described with examples where the wake-up command, authentication key, and communication key are all transmitted and received as acoustic signals; however, aspects of this disclosure should not be considered as limited to such a requirement. Rather, as indicated above, the wake-up command and the authentication key may be received as RF signals during the initialization phase.

In one or more the example techniques described in this disclosure, one or more sensors of the IMD, which are used to sense one or more patient characteristics, may sense the signal or signals transmitted by the external device during the initialization phase (e.g., the acoustic signals). For example, the IMD may include an accelerometer to sense patient position or movement. As another example, the IMD may include a pressure sensor to sense the pressure at one or more locations in the patient. In some examples, in addition to sensing patient characteristics, the IMD may utilize these one or more sensors for communication purposes. For example, the one or more sensors may sense the acoustic signal transmitted by the external device, and these same one or more sensors may sense patient characteristics such as patient position, patient movement, and pressure. Hence, a sensor may be, in effect, reused or used for two or more purposes, i.e., sensing one or more patient characteristics and sensing a signal to support secure telemetry.

In response to sensing the signal or signals transmitted during the initialization phase (e.g., the wake-up command, authentication key, and/or communication key), these one or more sensors may output an electrical signal. The IMD may retrieve the wake-up command, authentication key, and/or communication key from the electrical signal outputted by these one or more sensors. In this manner, the IMD may utilize these one or more sensors for sensing patient characteristics, as well as for communication purposes.

A secure telemetry system typically relies on a shared secret key for encryption and authentication. The channel that is used for actual data communication and authentication may be based on different communication methods for improved security. For example, magnetic field based telemetry may be used for authentication before radio frequency (RF)-based telemetry is used for data communication. However, this approach may require a proximal antenna and receiver/transmitter to be designed and implemented on both the IMD and an external device.

In accordance with some examples of this disclosure, a sensor within the IMD that senses patient characteristics may also sense a signal that includes a communication key. The IMD may then utilize the communication key that is included in the signal sensed by the sensor to decrypt received information and/or encrypt transmitted information. If a sensor provided in the IMD to sense one or more patient characteristics is reused to support secure telemetry, it may be possible to reduce complexity, size and/or cost of the IMD and/or the external device.

As an example, an authentication technique based on an acoustic signal (e.g., an acoustic signature waveform such as a predefined tone sequence) may be used for authentication and/or wakeup. In some examples, the acoustic receiver, in the IMD, may reuse an accelerometer or other sensor already provided in the IMD to sense one or more patient characteristics. For example, an accelerometer used in the IMD for patient posture detection may be used to detect audio tone from an external device. The accelerometer senses one or more patient characteristics such as patient posture or activity, but also can be used to sense an acoustic signal to support secure telemetry.

Authentication and/or wakeup can be performed, for example, once a predetermined audio sequence is detected by the IMD using an acoustic sensor such as the accelerometer. This technique may allow shared secret data exchange to occur, thereby supporting secure primary communication method, e.g., via radio frequency (RF) telemetry. An audio sequence generated by the external device can also be updated once a secure primary communication channel has been established. Use of acoustic sensor may provide added flexibility for performing authentication for a primary telemetry system. Although an acoustic sensor may be used, in some examples, more generally, a sensor within the IMD that senses patient characteristics may also sense a signal, such as an acoustic signal, that includes a communication key.

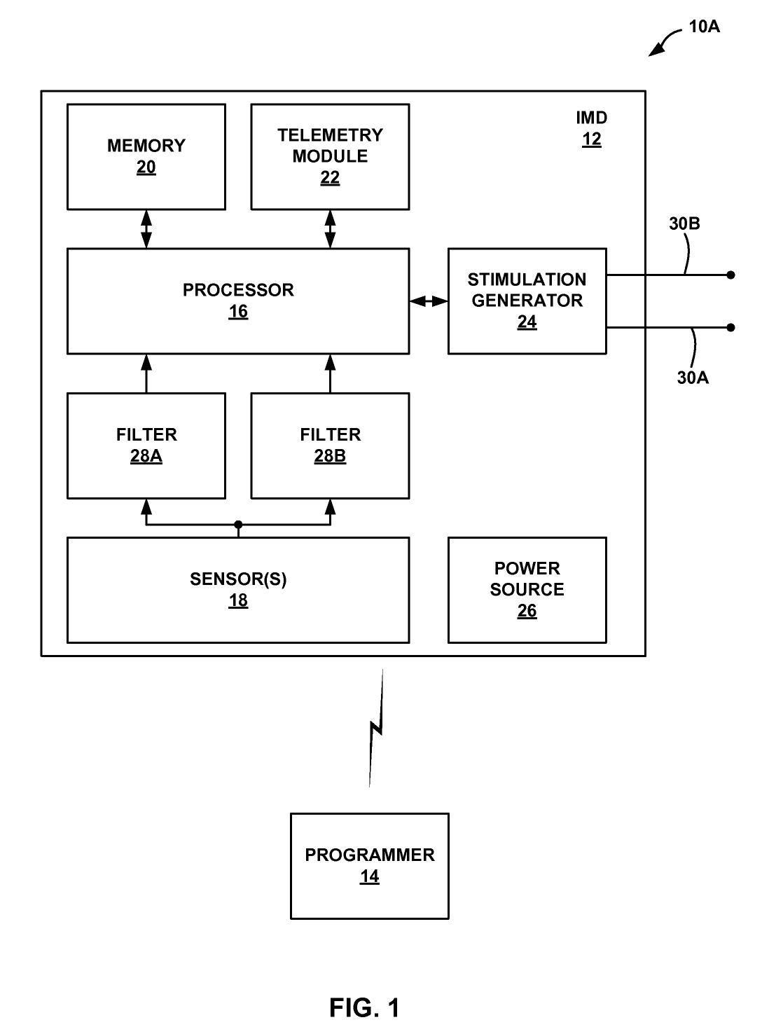

FIG. 1 is a block diagram illustrating an implantable medical device (IMD) of a system that includes the IMD and an external device. For example, as illustrated in FIG. 1, system 10A includes IMD 12 and programmer 14. Programmer 14 is an example of an external device. For instance, an external device may be any device that is not implanted within the patient.

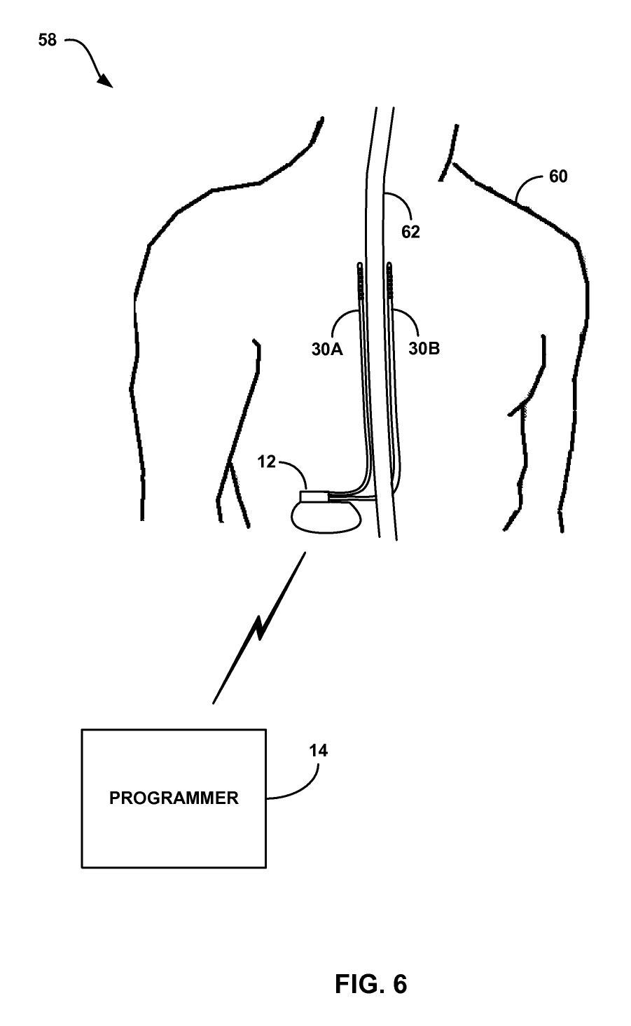

IMD 12 may deliver therapy to the patient within whom IMD 12 is implanted. An example of IMD 12 implanted within the patient is illustrated in further detail in FIG. 6. For instance, IMD 12 may deliver electrical stimulation therapy to the patient, deliver drug therapy, or deliver both electrical stimulation and drug therapy. As illustrated, IMD 12 may include processor 16, one or more sensors 18, memory 20, telemetry module 22, stimulation generator 24, and power source 26. As described in more detail, IMD 12 may also include filters 28A and 28B in some examples. IMD 12 may include additional components that are not shown for purposes of clarity. For purposes of illustration, IMD 12 of FIG. 1 is illustrated as providing electrical stimulation therapy (e.g., via stimulation generator 24); however, IMD 12 may also deliver drug therapy via a drug delivery module (not shown) in addition to or instead of delivering electrical stimulation. In some examples, IMD 12 may be dedicated to monitor or sense patient characteristics, and may not actively deliver therapy.

Memory 20 may store instructions for execution by processor 16, stimulation therapy program data, sensor data, operational and status data, and any other information regarding therapy or the patient. Stimulation program data may include stimulation parameters transmitted from programmer 14, as well as programs defined by such parameters, and program groups. Some data may be recorded for long-term storage and retrieval by a user. Memory 20 may include separate memories for storing different types of data.

Processor 16 controls stimulation generator 24 to deliver electrical stimulation via electrode combinations formed by electrodes in one or more electrode arrays. For example, stimulation generator 24 may deliver electrical stimulation therapy via electrodes of one or more leads 30A and 30B (collectively referred to as leads 30), e.g., as stimulation pulses or continuous waveforms. Stimulation generator 24 may include stimulation generation circuitry to generate stimulation pulses or waveforms and switching circuitry to switch the stimulation across different electrode combinations, e.g., in response to control by processor 16. For example, processor 16 may control the switching circuitry on a selective basis to cause stimulation generator 24 to deliver electrical stimulation to selected electrode combinations and to shift the electrical stimulation to different electrode combinations. Alternatively, in some examples, stimulation generator 24 may include multiple current or voltage sources to control delivery of stimulation energy to selected combinations of electrodes carried by leads 30.

For instance, in examples where IMD 12 provides electrical stimulation therapy, IMD 12 may be a cardiac stimulator that provides electrical stimulation therapy to the heart of the patient. In other example, IMD 12 may be a neurostimulator that provides spinal cord stimulation, deep brain stimulation, pelvic floor stimulation, gastric stimulation, peripheral nerve stimulation, and other examples where electrical stimulation therapy may be appropriate. In examples where IMD 12 is a cardiac stimulator, programmer 14 (which is described in more detail below) may be a physician programmer, and the patient may not be allowed to interact with programmer 14. In examples where IMD 12 is a neurostimulator, programmer 14 may be a physician programmer or a patient programmer. A patient programmer may provide the patient with some, limited interactive options. A physician programmer may be usable by a physician or a medically trained professional. The physician programmer may provide more functional options than the patient programmer because the physician programmer is generally operated by a physician or a medically trained professional, and the physician or the medically trained professional may ensure that the therapy provided by IMD 12 remains within safety parameters.

Electrode combinations and other parameters associated with different therapy programs may be represented by data stored in a memory location, e.g., in memory 20, of IMD 12. Processor 16 may access the memory location to determine the electrode combination for a particular program and control stimulation generator 24 to deliver electrical stimulation via the indicated electrode combination. Each program may specify a set of parameters for delivery of electrical stimulation therapy. As an example, a program may specify electrode combination, electrode polarities, current or voltage amplitude, pulse rate and pulse width. Additional parameters such as duty cycle, duration, and delivery schedule also may be specified by a therapy program.

Using an external programmer, such as programmer 14, a user may select individual programs for delivery on an individual basis, or combinations of programs for delivery on a simultaneous or interleaved basis. In addition, a user may adjust parameters associated with the programs. The programs may be stored in memory 20 of IMD 12. Alternatively, the programs may be stored in memory associated with external programmer 14. In either case, the programs may be selectable and adjustable to permit modification of therapy parameters. In addition, a physician programmer may permit generation of new programs, which may be loaded into memory 20, and adjustment of parameters associated with existing programs. In some examples, programmer 14 and IMD 12 may communicate with one another using secure communication.

Upon selection of a particular program or program group from memory 20, processor 16 may control stimulation generator 24 to deliver stimulation according to the programs in the groups, e.g., simultaneously or on a time-interleaved basis. A group may include a single program or multiple programs, each of which specifies an electrode combination. Again, the electrode combination may specify particular electrodes in a single array or multiple arrays, e.g., on a single lead or among multiple leads.

One or more sensors 18 may be sensors that sense patient characteristics. Examples of the one or more sensors 18 include accelerometers and pressure sensors. As one example, one or more sensors 18 may sense patient position (e.g., sitting, standing, prone, supine, etc.) and patient movement (e.g., walking, running, or still) in examples where one or more sensors 18 are accelerometers. As another example, one or more sensors 18 may sense pressure within the patient in examples where one or more sensors 18 are pressure sensors. For example, one or more sensors 18 may be coupled to a fluid tube that extends to the bladder of the patient. One or more sensors 18 may measure the bladder pressure by measuring the pressure of the fluid tube.

Although one or more sensors 18 are illustrated as being within IMD 12, aspects of this disclosure are not limited to such a requirement. In some alternate examples, leads 30A and/or 30B may include one or more sensors 18. In these alternate examples, one or more sensors 18 may be located in the distal end, proximal end, or anywhere in between the distal and proximal ends of leads 30A and/or 30B. In some other examples, at least one of sensors 18 may be located within IMD 12, and at least one of sensors 18 may be located within lead 30A or lead 30B.

Processor 16 may receive the sensed patient characteristics from one or more sensors 18 and utilize these sensed patient characteristics as inputs into the therapy program that processor 16 executes. For example, processor 16 may execute different therapy programs or a therapy program with different therapy parameters based on the position or movement of the patient or based on the pressure within the patient. In some examples, memory 20 may store the patient characteristics sensed by one or more sensors 18 for eventual transmission to programmer 14. A user of programmer 14 may utilize these sensed patient characteristics to program appropriate therapy programs. IMD 12 may be responsive to adjustments of programming parameters and electrode configurations by a user via programmer 14.

In alternate examples, the sensed patient characteristics may be used in a closed loop fashion to allow processor 16 to determine which therapy to provide. For example, processor 16 may receive the sensed patient characteristics from one or more sensors 18, such as a particular patient posture. Processor 16 may select the therapy program, among a plurality of therapy programs, that provides the appropriate therapy for the given patient posture, as sensed by one or more sensors 18. Even in these examples, the sensed patient characteristics may be transmitted to programmer 14. However, in these alternate examples, it may not be necessary for programmer 14 to select the specific therapy program that processor 16 should execute. Rather, processor 16 may select the therapy program based on the sensed patient characteristics as sensed by one or more sensors 18.

As indicated above, programmer 14 and IMD 12 may communicate information to one another such as programming parameters and electrode configurations from programmer 14 to IMD 12 and results of the therapy or sensed patient characteristics from IMD 12 to programmer 14 as a few examples. Communication between programmer 14 and IMD 12 may include an initialization phase and a transfer phase. In the initialization phase, IMD 12 may ready itself for receiving information from and/or transmitting information to programmer 14. In the transfer phase, IMD 12 and programmer 14 may transmit information to and/or receive information from one another.

In the initialization phase, programmer 14 may transmit a wake-up command and an authentication key. The wake-up command may cause IMD 12 to transition from a dormant mode or sleep mode to a communication mode (e.g., a mode where IMD 12 is ready to receive and/or transmit information). The wake-up command transmitted by programmer 14 may be a simple acoustic “ping,” or a more complicated digital bit stream represented by a sequence of audio tones, as described in more detail.

The authentication key may be a unique identifier of programmer 14. For example, each programmer may be assigned its own unique 8-bit authentication key, and programmer 14 may transmit its unique 8-bit authentication key to IMD 12 during the initialization phase, as one example. The 8-bit authentication key is provided for illustration purposes only. In general, programmer 14 may transmit any sized authentication key that uniquely identifies programmer 14. IMD 12 may utilize the authentication key to determine whether programmer 14 is an external device with which IMD 12 is authorized to communicate. For example, memory 20 may store one or more authentication keys, and processor 16 may receive the authentication key from programmer 14, as described in more detail, and determine whether the authentication key matches any one of the authentication keys stored in memory 20 to determine whether IMD 12 is authorized to communicate with programmer 14.

The wake-up command and the authentication key may be optional in the initialization phase. For instance, if IMD 12 is always in communication mode, then the wake-up command may not be necessary. Also, if IMD 12 is authorized to communicate with any programmer, the authentication key may not be necessary. However, for reduction in power consumption and for security purposes, it is generally suitable for programmer 14 to transmit the wake-up command and the authentication key during the initialization phase.

In examples described in this disclosure, during the initialization phase, programmer 14 may transmit a communication key to IMD 12. IMD 12 may utilize the communication key to code information received in the transfer phase or transmitted in the transfer phase. For example, IMD 12 may decrypt (e.g., decode) information received in the transfer phase, or encrypt (e.g., encode) the information that IMD 12 transmits in the transfer phase. The term “code” or “coding” is used in this disclosure to refer to encrypting and decrypting for purposes of brevity. For example, the terms encrypt and encode may be used interchangeably. Similarly, the terms decrypt and decode may be used interchangeably. Encrypting, encoding, decrypting, and decoding may be commonly referred to as coding for purposes of brevity. In some examples, programmer 14 may transmit a new communication key for every communication instantiation. IMD 12 and programmer 14 may use the new communication key to code information for every communication instantiation.

As described in more detail, IMD 12 may utilize one or more sensors 18 to receive the information transmitted during the initialization phase. In other words, IMD 12 may utilize one or more sensors 18 for sensing patient characteristics. IMD 12 may utilize these same one or more sensors 18 for sensing the signal that includes the communication key in the initialization phase. In some examples, IMD 12 may utilize these same one or more sensors 18 for sensing the signal or signals that include the wake-up command and the authentication key in the initialization phase.

During the transfer phase, IMD 12 may receive information from programmer 14. For example, the received information may include configuration parameters, therapy programs, updates to the authentication key, updates to the wake-up command, or other such information. Because information received by IMD 12 may be personal and sensitive information and may be particularized for IMD 12, programmer 14 may code, e.g., encode or encrypt, the information such that only IMD 12 can decrypt or decode the received information. For example, programmer 14 may encrypt the information in such a manner that the information can only be decrypted with the communication key. IMD 12 may then utilize its received communication key, via one or more sensors 18, to code, e.g., decode or decrypt, the encoded information.

In the reverse, during the transfer phase, IMD 12 may code, e.g., encode or encrypt, information that IMD 12 transmits to programmer 14 such that the encoded information can only be decoded with the communication key. The information that IMD 12 transmits to programmer 14 may include patient information, therapy results, sensed patient characteristics, or other such information. Programmer 14 may decode the received information using the communication key.

As illustrated in FIG. 1, IMD 12 includes telemetry module 22. IMD 12 may utilize telemetry module 22 to transmit and receive information during the transfer phase. Telemetry module 22 may support wireless telemetry with external programmer 14 or another device by radio frequency (RF) communication, as one example. For example, telemetry module 22 may transmit and receive RF signals. Telemetry module 22 may send information to and receive information from external programmer 14 on a continuous basis, at periodic intervals, or upon request from IMD 12 or programmer 14. To support RF communication, telemetry module 22 may include appropriate electronic components, such as amplifiers, filters, mixers, encoders, decoders, modulators, demodulators and the like. The frequency of the RF signal may be in the range of approximately 1 MHz to 2.4 GHz, although aspects of this disclosure are not so limited. For example, the RF signal may be approximately within the 402-405 MHz band, or at 2.4 GHz.

With the communication key, IMD 12 and programmer 14 may securely communicate with one another. In this manner, a device other than programmer 14 may not be able to decipher the information transmitted by IMD 12 because this other device would not have access to the communication key, and would, therefore, not be able to decode the information transmitted by IMD 12. Such secure communication may protect patient privacy, as well as lower the probability of an IMD, other than IMD 12, inadvertently performing functions in response to a transmission from programmer 14.

For example, an IMD, other than IMD 12, may happen to be in the vicinity of IMD 12 (e.g., in a clinic or hospital setting where multiple patients are nearby, or where the patient is implanted with multiple IMDs), and may receive the information transmitted by programmer 14, along with IMD 12. Because the information transmitted by programmer 14 may be particularized to IMD 12, it may be undesirable for the other IMD to perform functions in response to the transmission by programmer 14. In accordance with the techniques of this disclosure, the other IMD may not respond to the transmission by programmer 14 because the other IMD may not have access to the communication key that is particular to the communication between IMD 12 and programmer 14, and, therefore, would not be able to decode the transmitted information.

Furthermore, using a single communication key for coding purposes is provided for example purposes and should not be considered limiting. For example, in some instances, programmer 14 may transmit a decode communication key and an encode communication key, which may be different communication keys. IMD 12 may utilize the decode communication key to decode the information received in the transfer phase, and utilize the encode communication key to encode the information transmitted in the transfer phase. In either example, IMD 12 receives the communication key or keys.

In some examples, IMD 12 and programmer 14 may utilize different communication techniques during the initialization phase and the transfer phase. As one example, IMD 12 may not utilize telemetry module 22 to receive one or more signals from programmer 14 that include the communication key, wake-up command, and authentication key. In this example, IMD 12 may utilize one or more sensors 18 to sense the one or more signals from programmer 14 that include the communication key, wake-up command, and authentication key. IMD 12 may utilize telemetry module 22 to receive and transmit one or more signals (e.g., RF signals) during the transfer phase.

In some alternate examples, IMD 12 may utilize telemetry module 22 to receive the wake-up command and the authentication key during the initialization phase. For example, programmer 14 may transmit the wake-up command and the authentication key using RF signals that are received by telemetry module 22. In these alternate examples, IMD 12 may not utilize telemetry module 22 to receive the communication key during the initialization phase, and may communicate with programmer 14 using telemetry module 22 during the transfer phase. For purposes of brevity, the examples described below are described in context where IMD 12 receives the wake-up command, authentication key, and communication key without using RF signals (e.g., without using telemetry module 22); however, as discussed above, aspects of this disclosure are not so limited, and IMD 12 may receive the wake-up command and authentication key as RF signals (e.g., using telemetry module 22).

The signal or signals that IMD 12 receives during the initialization phase may be acoustic signals. For example, IMD 12 may receive one or more acoustic signals that include the communication key, the wake-up command, and the authentication key. For instance, the acoustic signal may be a digital bit stream where a digital one is represented by a first acoustic tone and a digital zero is represented by a second acoustic tone. The frequency of the first and second acoustic tones may be different. In some examples, the second acoustic tone may be a “no tone.” The frequency of the first and second acoustic tones may be in the range of approximately 20 Hertz (Hz) to 20 kHz, although aspects of this disclosure are not so limited.

For example, during the initialization phase, a user of programmer 14 may place programmer 14 in close proximity to the patient. For instance, programmer 14 may include an acoustic head that is placed over IMD 12. During the initialization phase, programmer 14 may transmit the one or more acoustic signals that include the communication key, the wake-up command, and the authentication key. Due to the close proximity of programmer 14 to the patient, the chances of an IMD, other than IMD 12, receiving the one or more acoustic signals, during the initialization phase, may be minimized. For example, one or more sensors 18 may be able to sense acoustic signals of a certain amplitude. If programmer 14 is too far from the patient, the amplitude of the acoustic signals transmitted by programmer 14, during the initialization phase, may become too attenuated for one or more sensors 18 to sense. In this manner, one or more sensors 18 may not be able to sense acoustic signals that are transmitted by programmers, other than programmer 14, because these other programmers may not be in the vicinity of IMD 12, and the acoustic signals of these other programmer may become too attenuated to be sensed by one or more sensors 18.

As one example, the communication key may be a 128-bit communication key. The 128-bit communication key may include a plurality of digital ones and a plurality of digital zeros. In this example, for each of the plurality of digital ones, programmer 14 may transmit an acoustic tone at a first frequency and for each of the plurality of digital zeros, programmer 14 may transmit a tone at a second, different frequency. It should be understood that the frequency of the bit stream need not be in the acoustic frequency range. Rather, in this example, the digital ones and zeros of the bit stream are represented by acoustic tones at different frequencies.

As another example, the acoustic signal may be a phase or pulse modulated signal. For example, programmer 14 may phase modulate a carrier wave with two acoustic modulation frequencies. In this example, a first acoustic modulation frequency may represent a digital one, and a second acoustic modulation frequency may represent a digital zero. The second acoustic modulation frequency may be “no modulation.” The frequency of the first and second acoustic modulation frequencies may be in the range of approximately 20 Hz to 20 kHz, although aspects of this disclosure are not so limited.

Programmer 14 may similarly transmit the wake-up command and the authentication key. For example, during normal mode of operation, processor 16 may place telemetry module 22 in dormant or sleep mode to reduce power consumption. Programmer 14 may transmit an acoustic signal that includes the wake-up command, and in response, processor 16 may transition telemetry module 22 into a communication mode so that telemetry module 22 is capable of transmitting and receiving information during the transfer phase.

However, in examples where IMD 12 receives the wake-up command and authentication key via telemetry module 22, processor 16 may not place telemetry module 22 in full dormant or sleep mode, but rather in partial dormant or sleep mode so that telemetry module 22 can receive the wake-up and authentication key when necessary. Even in full dormant or sleep mode or partial dormant or sleep mode, telemetry module 22 may be able to perform some limited functions. Alternatively, in full dormant or sleep mode, telemetry module 22 may be non-functional.

Programmer 14 may transmit a single tone (e.g., a ping sound) as the wake-up command. However, to avoid extraneous noise from inadvertently waking-up telemetry module 22, the wake-up command may include a digital bit stream of digital ones and zeros, similar to the communication key, but the number of digital bits may not need to be as large as the communication key. For example, a digital bit stream of 8-bits may be sufficient for the wake-up command. In this example, memory 20 may store a wake-up command. Processor 16 may compare the received wake-up command to the stored wake-up command, and cause telemetry module 22 to wake-up when the received wake-up command is equivalent to the stored wake-up command.

After waking-up, processor 16 may receive the authentication key as part of an acoustic signal transmitted by programmer 14 that is sensed by one or more sensors 18. As described above, processor 16 may compare the authentication key to stored authentication keys, and based on the comparison allow IMD 12 and programmer 14 to communication with one another. Processor 16 may then receive the communication key and code information that is transmitted by or received by telemetry module 22 (e.g., the RF signal) using the communication key during the transfer phase.

In some examples, processor 16 may accept the communication key when processor 16 verifies that authentication key is one of the stored authentication keys. For example, if the received authentication key is not one of the authentication keys stored in memory 20, then IMD 12 may not be authorized to communicate with the programmer that transmitted the authentication key. To avoid IMD 12 from communicating with this unauthorized programmer, processor 16 may not accept the communication key that is transmitted by this unauthorized programmer.

As described above, one or more sensors 18 may sense the information transmitted by programmer 14 during the initialization phase (e.g., the communication key). As one example, one of one or more sensors 18 may be an accelerometer. The signal transmitted by programmer 14 during the initialization phase (e.g., the acoustic signal) may excite the accelerometer, which in turn causes the accelerometer to output an electrical signal that corresponds to the acoustic signal. As another example, one of the one or more sensors 18 may be a pressure sensor. The acoustic signal transmitted by programmer 14 during the initialization phase may cause the pressure sensor to detect a change in pressure, which in turn causes pressure sensor to output an electrical signal that corresponds to the acoustic signal.

In this manner, one or more sensors 18 may sense a signal transmitted by programmer 14 during the initialization phase, which may be an acoustic signal that includes one or more of the wake-up command, authentication key, and communication key. One or more sensors 18 may not be capable of sensing signals transmitted during the transfer phase. For example, one or more sensors 18 may not be able to sense the high frequencies of the RF signals transmitted during the transfer phase.

The electrical signal outputted by one or more sensors 18 may represent the wake-up command, authentication key, and/or communication key. Processor 16 may receive the outputted electrical signals and may perform signal processing to extract the wake-up command, authentication key, and communication key. For example, in examples where the acoustic signal is phase modulated, processor 16 may demodulate the signal and extract the wake-up command, authentication key, and communication key. As described above, processor 16 may compare the wake-up command and authentication key to corresponding commands and keys stored in memory 20. Processor 16 may also store the communication key in memory 20 for when processor 16 codes information transmitted by or received by telemetry module 22. Processor 16 may update the communication key for every communication instantiation.

For example, programmer 14 may create a new communication key for every instance that is communicates with IMD 12. Programmer 14 may transmit the newly created communication key (e.g., for every communication instantiation) to IMD 12 via the acoustic signal or signals. In response, processor 16 may updated the communication key to the newly received communication key and use the new communication key for communicating with programmer 14 for the current communication instantiation.

Processor 16 may be programmed to accept the communication key within a defined time period after receiving the authentication key in some examples. For example, as discussed above, processor 16 may confirm that the received authentication key corresponds to one of the stored authentication keys. After processor 16 confirms that IMD 12 is authorized to communicate with the programmer that transmitted the authentication key (e.g., programmer 12 in this example), there may be a defined period within which IMD 12 receives the communication key. If processor 16 determines that IMD 12 did not receive the communication key within the defined period, processor 16 may place telemetry module 22 back into full or partial dormant or sleep mode, and not accept any communication key that is received after the defined period without first receiving another authentication key. This may further protect IMD 12 from communicating with unintended external devices. A defined period within which IMD 12 receives the communication key after receiving the authentication key is not required in every example.

In some examples, programmer 14 may update the authentication key and/or wake up command. For example, during the transfer phase, programmer 14 may transmit an RF signal that updates its authentication key. In response, processor 16 may update the authentication keys listed in memory 20. As another example, during the transfer phase, programmer 14 may transmit an RF signal that updates the wake-up command. In response, processor 16 may update the wake-up command stored in memory 20.

Because one or more sensors 18 may output an electrical signal in response to sensing patient characteristics and in response to sensing information transmitted during the initialization phase, processor 16 may determine whether a current electrical signal outputted by one or more sensors 18 is for sensed patient characteristics or for sensed information transmitted by programmer 14. To assist in this determination, IMD 12 may include filters 28A and 28B. In this example, filter 28A may be low-pass filter and filter 28B may be high-pass filter.

Filters 28A and 28B may be digital filters or analog filters. In examples where filters 28A and 28B are digital filters, one or more sensors 18 may include or may be coupled to an analog-to-digital converter that converts the analog signals outputted by one or more sensors 18 into digital signals for filtering by filters 28A and 28B. In examples where filters 28A and 28B are digital filters or analog filters, one or more sensors 18 may include or may be coupled to amplifiers that amplify the signal generated by one or more sensors 18 prior to being filtered by filters 28A and 28B.

Also, as discussed above, in some examples, lead 30A and/or lead 30B may include one or more sensors 18. In these examples, one or more sensors 18, located within lead 30A and/or 30B, may be coupled to filters 28A and 28B. In other words, in these examples, IMD 12 may include wiring that extends from filters 28A and 28B, located within IMD 12, to one or more sensors 18 located within lead 30A and/or 30B. It may be possible for filters 28A and 28B to be located within leads 30A and/or 30B, instead of IMD 12, in examples where one or more sensors 18 are located within leads 30A and/or 30B.

In some examples, the electrical signals outputted by one or more sensors 18 in response to sensing a patient characteristic may be at a lower frequency than the electrical signals outputted by one or more sensors 18 in response to sensing information transmitted by programmer 14 (e.g., one or more of the wake-up command, authentication key, and communication key). In this example, when one or more sensors 18 sense a patient characteristic, the electrical signals outputted by one or more sensors 18 may pass through low-pass filter 28A and may be blocked by high-pass filter 28B. Also, in this example, when one or more sensors 18 sense information transmitted by programmer 14, the electrical signals outputted by one or more sensors 18 may pass through high-pass filter 28B and may be blocked by low-pass filter 28A.

Processor 16 may generally refer to processing circuitry, and may include two amplitude measurement modules, each coupled to one of filter 28A and filter 28B. When the amplitude measurement module coupled to filter 28A detects a signal from filter 28A (e.g., the amplitude of the signal is sufficiently high), processor 16 may recognize that one or more sensors 18 sensed patient characteristics. When the amplitude measurement module coupled to filter 28B detects a signal from filter 28B, processor 16 may recognize that one or more sensors 18 sensed information transmitted by programmer 14. In this manner, processor 16 may determine whether electrical signals received from one or more sensors 18 are part of the therapy operation of IMD 12 or part of the communication operation of IMD 12.

Determining whether one or more sensors 18 sensed patient characteristics or information transmitted by programmer 14 utilizing filters 28A and 28B is provided for illustration purposes only and should not be considered limiting. Processor 16 may utilize any technique to differentiate between the sensed patient characteristic signals and sensed information signals outputted by one or more sensors 18.

Utilizing one or more sensors 18 for sensing patient characteristics and for sensing a signal transmitted by programmer 14 may reduce the number of components of IMD 12. For example, conventional IMDs received wake-up commands, authentication keys, and communication keys via a magnetic field. To receive the magnetic field, the conventional IMDs included a communication coil which used up available space within the IMDs.

In the example of FIG. 1, IMD 12 may not require a communication coil to receive wake-up commands, authentication keys, and communication keys via a magnetic field. As described above, one or more sensors 18, which are usable for sensing patient characteristics, may also sense signals that include wake-up commands, authentication keys, and communication keys. Not including a communication coil in IMD 12 may reduce the size of IMD 12 which may be desirable for implantation purposes.

Power source 26 delivers operating power to the components of IMD 12. Power source 26 may include a small rechargeable or non-rechargeable battery and a power generation circuit to produce the operating power. Recharging may be accomplished through proximal inductive interaction between an external charger and an inductive charging coil within IMD 12. In some examples, power requirements may be small enough to allow IMD 12 to utilize patient motion and implement a kinetic energy-scavenging device to trickle charge a rechargeable battery. In other examples, non-rechargeable batteries may be used for a limited period of time. As a further alternative, an external inductive power supply could transcutaneously power IMD 12 when needed or desired.

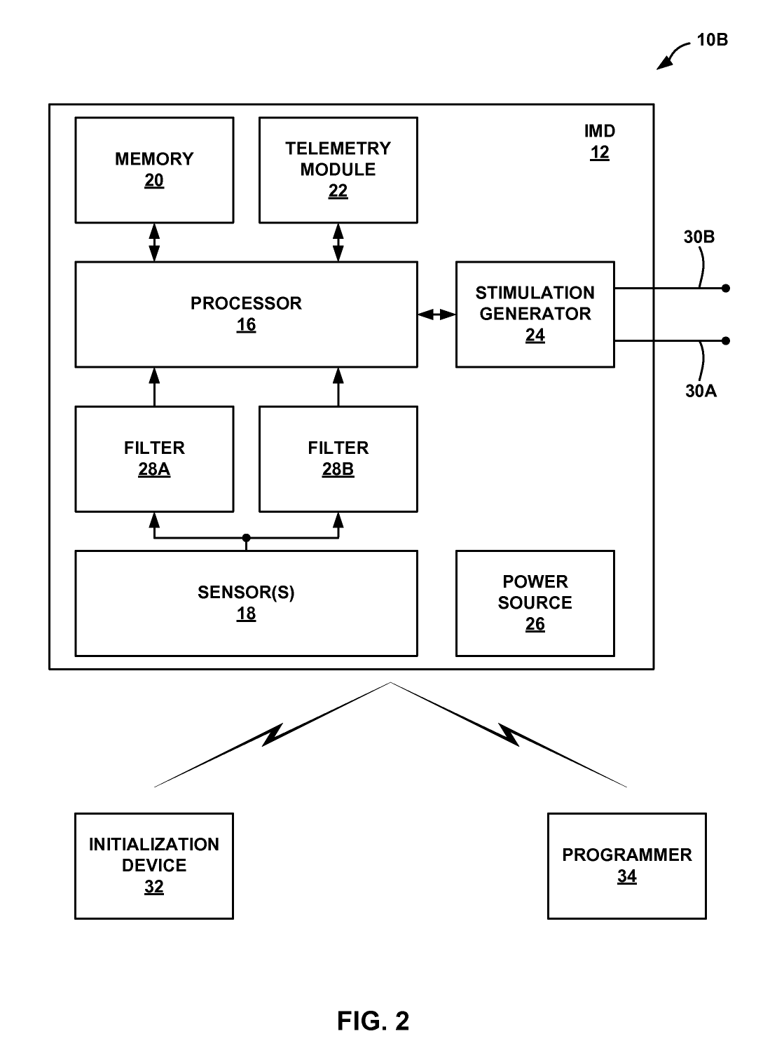

Although FIG. 1 illustrates IMD 12 communicating with only programmer 14 during both the initialization phase and the transfer phase, aspects of this disclosure are not so limited. In some alternate examples, IMD 12 may communicate with one device during the initialization phase and communicate with another device during the transfer phase. Such an alternate example is illustrated in further detail with respect to FIG. 2.

FIG. 2 is a block diagram illustrating an implantable medical device (IMD) of a system that includes the IMD and two external devices. For example, as illustrated in FIG. 2, system 10B includes IMD 12, initialization device 32, and programmer 34. For purposes of brevity, only the components that are different between FIG. 1 and FIG. 2 are described in greater detail.

The example of FIG. 2 may be applicable to settings where the patient visits a clinic, office, or hospital. In the example of FIG. 2, programmer 34 may be stand-alone computer, and it may be difficult to transport programmer 34. For example, programmer 14 of FIG. 1 may be a handheld device that a user can easily carry, whereas the user may not be able to carry programmer 34. In the example of FIG. 2, initialization device 32 may be a handheld device that a user can easily carry.

In system 10B, when the patient visits the clinic, office, or hospital, a clinician or physician may couple initialization device 32 to programmer 34 via a communication cable or wirelessly. Programmer 34 may transmit the communication key and its authentication key to initialization device 32. The clinician or physician may disconnect initialization device 32 from programmer 34, and place initialization device 32 in proximity to the patient.

Initialization device 32 may then transmit the wake-up command, authentication key, and the communication key, as described above. For example, the physician or clinician may place initialization device 34 in close proximity to the patient and in some examples over IMD 12. Initialization device 34 may transmit the one or more acoustic signals, of the initialization phase, that include the wake-up command, authentication key, and the communication key. In this example, initialization device 32 may transmit the authentication key for programmer 34 because IMD 12 will communicate with programmer 34 during the transfer phase. Also, because initialization device 32 is in proximity to the patient, the likelihood of another device receiving the information transmitted by initialization device 32, during the initialization phase, to IMD 12 is minimized.

Programmer 34 and IMD 12 may then communicate with one another as described above. For example, telemetry module 22 may receive coded information from or transmit coded information to programmer 34 using RF signals. Processor 16 may code the information using the communication key. Also, by coding information using the communication key, the information transmitted by IMD 12 may only be decoded by programmer 34, and not some other device. Similarly, by coding information using the communication key, the information transmitted by programmer 34 may only be decoded by IMD 12, and not some other device.

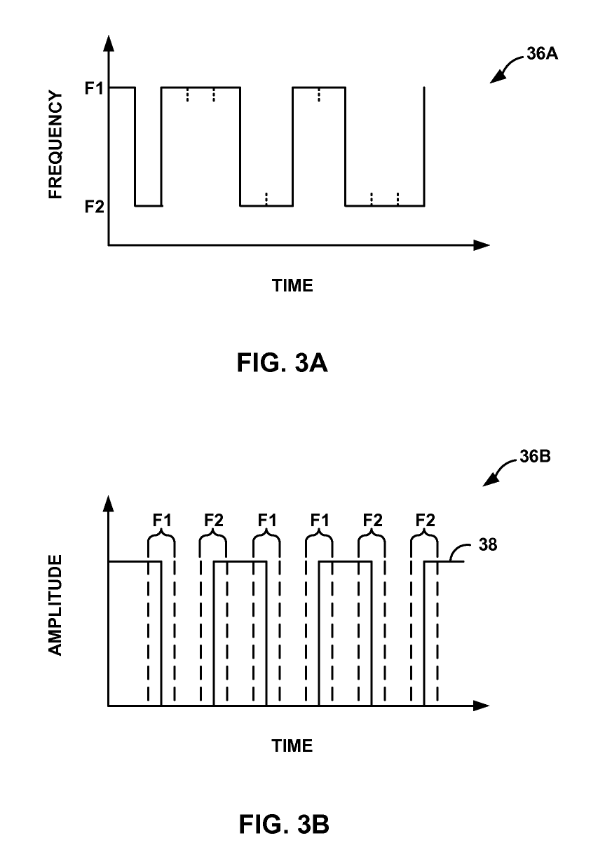

FIGS. 3A and 3B are timing diagrams illustrating examples of acoustic signals. For example, FIG. 3A illustrates acoustic signal 36A, and FIG. 3B illustrates acoustic signal 36B. Acoustic signals 36A and 36B may be examples of acoustic signals that include communication keys. Programmer 14 (FIG. 1) or initialization device 32 (FIG. 2) may transmit acoustic signals 36A or 36B during the initialization phase. Furthermore, although the acoustic signals of FIGS. 3A and 3B are described as being those for communication keys, programmer 14 or initialization device 32 may transmit the acoustic signals for the wake-up command and the authentication key in a substantially similar manner.

In the example of FIG. 3A, the communication key may be a “101110011000” bit stream. For example, the dashed lines in FIG. 3A illustrate the temporal width of a single bit. In this example, a digital one is represented by a tone with acoustic frequency F1, and a digital zero is represented by a tone with acoustic frequency F2. Frequency F1 and F2 may be different and within the range of approximately 20 Hz to 20 kHz, although aspects of this disclosure are not so limited. Also, the frequency of acoustic signal 36A need not be within 20 Hz to 20 kHz, but it may possible for this to be the case. In some examples, the frequency of acoustic signal 36A may be approximately 500 Hz to 1.5 kHz; however, aspects of this disclosure are not so limited.

In the example of FIG. 3B, acoustic signal 36B may be a phase modulated acoustic signal. For example, carrier wave 38 may be phase modulated with a modulation acoustic frequency of F1 or a modulation acoustic frequency of F2. Similar to FIG. 3A, a modulation acoustic frequency of F1 may represent a digital one and a modulation acoustic frequency of F2 may represent a digital zero. In the example of FIG. 3B, the communication key may be a “101100” bit stream. In some examples, the frequency of carrier wave 38 may be approximately 100 Hz to 100 kHz; however, aspects of this disclosure are not so limited.

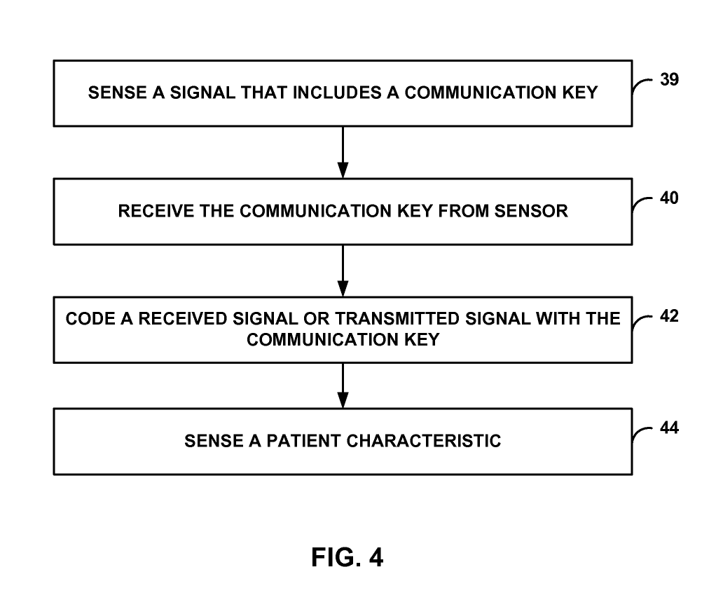

FIG. 4 is a flowchart illustrating an example operation of an IMD. For purposes of illustration only, reference is made to FIGS. 1-3B. One or more sensors 18 of IMD 12 may sense a first signal that includes a communication key (39). For example, an external device such as programmer 14 or initialization device 32 may transmit acoustic signal 36A or acoustic signal 36B. As described above, acoustic signal 36A may include a plurality of digital ones represented by a tone at a first acoustic frequency (e.g., F1), and a plurality of digital zeros represented by a tone at a second acoustic frequency (e.g., F2). Acoustic signal 36B may include a carrier wave 38 modulated at a first acoustic frequency (e.g., F1) that represents a digital one, and at a second acoustic frequency that represents a digital zero (e.g., F2).

In this example, one or more sensors 18 may sense acoustic signal 36A or acoustic signal 36B. For instance, in examples where one of sensors 18 is an accelerometer, acoustic signal 36A or acoustic signal 36B may excite the accelerometer and cause the accelerometer to output an electrical signal. In examples where one of sensors 18 is a pressure sensor, acoustic signal 36A or acoustic signal 36B may apply pressure that is detected by the pressure sensor.

Processor 16 of IMD 12 may receive the communication key from one or more sensors 18 (e.g., from the sensed first signal) (40). For example, in response to sensing the first signal, one or more sensors 18 may output an electrical signal that represents the communication key. Processor 16 may receive the electrical signal and perform signal processing to extract the communication key from the received electrical signal. In some examples, prior to processor 16 receiving the communication key from one or more sensors 18, filters 28A and 28B may filter the electrical signal outputted by one or more sensors 18. Filters 28A and 28B may assist processor 16 from differentiating between sensed patient characteristics and sensed first transmitted by an external device.

Processor 16 may code a second signal that is transmitted by telemetry module 22 of IMD 12, or code a second signal that is received by telemetry module 22 with the communication key received from one or more sensors 18 (42). For example, telemetry module 22 may receive a radio frequency signal (RF) signal from programmer 14 or programmer 34. The received RF signal may be encoded with the communication key. In this example, processor 16 may decode the received RF signal using the communication key. As another example, processor 16 may encode a signal that is to be transmitted. Telemetry module 22 may convert the encoded signal to an encoded RF signal and transmit the encoded RF signal to programmer 14 or programmer 34.

In examples of this disclosure, one or more sensors 18, which sensed the first signal, may also sense a patient characteristic (44). For example, one or more sensors 18 may sense patient position and patient movement. As another example, one or more sensors 18 may sense pressure within the patient.

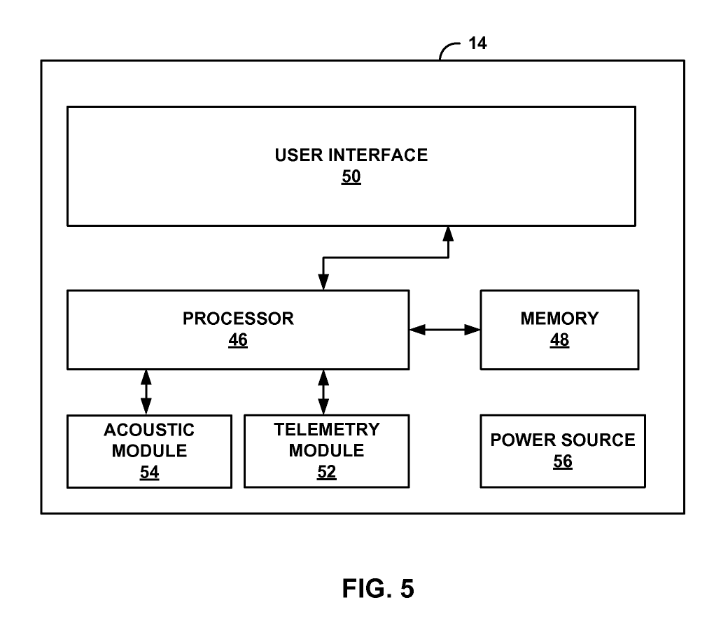

FIG. 5 is a block diagram illustrating an example external device. For example, FIG. 5 illustrates programmer 14, which is an example of an external device. As shown in FIG. 5, programmer 14 includes processor 46, memory 48, user interface 50, telemetry module 52, acoustic module 54, and power source 56. In the case of a clinician programmer, a clinician interacts with user interface 50 in order to generate programs and adjust program parameters, such as voltage or current amplitude, pulse width, pulse rate, electrode combinations and electrode polarities. Generation of programs and adjustment of program parameters may be aided by automated programming algorithms that guide the physician or clinician to select particular programs and program parameters. In the case of a patient programmer, a patient interacts with user interface 50 to select programs and adjust program parameters, e.g., on a limited basis as specified by the physician or clinician.

User interface 50 may include a screen and one or more input buttons that allow programmer 14 to receive input from a user. The screen may be a liquid crystal display (LCD), touch screen, or the like. The input buttons may include a touch pad, increase and decrease buttons, emergency shut off button, and other buttons needed to control the stimulation therapy. In some cases, the user may interact with user interface 50 via a stylus, soft keys, hard keys, directional devices, and any of a variety of other input media. In some examples, user interface 50 may be a keyboard or mouse.

Processor 46 receives input from user interface 50, presents data via the user interface 50, retrieves data from memory 48 and stores data within memory 48. Processor 46 also controls the transmission of information via telemetry acoustic module 54 and telemetry module 52. Memory 48 may include operational instructions for processor 46 or program parameter sets.

In examples of this disclosure, processor 46 may cause acoustic module 54 to transmit a first signal (e.g., an acoustic signal) to IMD 12. This acoustic signal may include one or more of a wake-up command, an authentication key of programmer 14, and a communication key selected by programmer 14. For example, programmer 14 may cause acoustic module 54 to transmit either acoustic signal 36A (FIG. 3A) or 36B (FIG. 3B). Acoustic module 54 may include an acoustic head that may be placed in proximity of the patient during the initialization phase. In this manner, one or more sensors 18 may sense the acoustic signal or signals transmitted by acoustic module 54, and the chances of one or more sensors 18 sensing the acoustic signal or signals transmitted by an acoustic module, other than acoustic module 54, may be minimized.

Telemetry module 52 allows the transfer of data to and from IMD 12. For example, telemetry module 52 may be similar to telemetry module 22 of IMD 12. Power source 56 may be a rechargeable battery, such as a lithium ion or nickel metal hydride battery. Other rechargeable or conventional batteries may also be used. In some cases, programmer 14 may be used when coupled to an alternating current (AC) outlet, i.e., AC line power, either directly or via an AC/DC adapter.

Programmer 34 of FIG. 2 may include components similar to those of programmer 14. However, in some examples, programmer 34 may not include acoustic module 54. In these examples, initialization device 32 may include a module similar to acoustic module 54. Also, in these examples, programmer 34 may transmit the wake-up command, its authentication key, and its communication key to initialization device 32. Initialization device 32 may then utilize its acoustic module 54 to transmit a signal that includes the wake-up command, authentication key, and communication key to IMD 12.

FIG. 6 is a schematic diagram illustrating an implantable medical device (IMD) implanted within a patient. As shown in FIG. 6, system 58 includes an implantable device 12 and programmer 14 shown in conjunction with patient 60. Although FIG. 6 shows IMD 12 coupled to fully implanted leads 30A, 30B, the techniques described in this disclosure may be applied to external stimulators coupled to leads via percutaneous lead extensions. Also, in some examples, implanted leads 30A and 30B may include one or more sensors 18 and filters 28A and 2B.

As shown in FIG. 6, leads 30A, 30B are implanted adjacent a spinal cord 62 of patient 60, e.g., for spinal cord stimulation (SCS) to alleviate pain. However, the techniques described in this disclosure are applicable to leads implanted to target any of a variety of target locations within patient 60, such as leads carrying electrodes located proximate to spinal cord 62, pelvic nerves, peripheral nerves, the stomach or other gastrointestinal organs, or within the brain of a patient. Also, techniques of this disclosure are applicable to other IMDs, such as those that deliver substances, e.g., drugs, to a patient.

In the example of FIG. 6, stimulation energy is delivered from device 12 to spinal cord 62 of patient 60 via one or more electrodes carried by axial leads 30A and 30B (collectively “leads 30”) implanted within patient 60. In various applications, such as spinal cord stimulation (SCS), the adjacent implantable leads 30 may have longitudinal axes that are substantially parallel to one another. Various combinations of electrodes carried by the leads 30 may be used to deliver electrical stimulation, including combinations of electrodes on a single lead or combinations of electrodes on both leads. Also, in some examples, electrodes may be carried by paddle leads in which an array of electrodes may be arranged in a two-dimensional pattern, e.g., as columns or rows of electrodes, on a common planar lead surface.

In the example of FIG. 6, leads 30 carry electrodes that are placed adjacent to the target tissue of spinal cord 62. In particular, leads 30 may be implanted in the epidural space adjacent spinal cord 62, and coupled to IMD 12. In the example of FIG. 6, stimulation energy may be delivered to spinal cord 62 to eliminate or reduce pain perceived by patient 60. However, IMD 12 may be used with a variety of different therapies, such as peripheral nerve stimulation (PNS), peripheral nerve field stimulation (PNFS), deep brain stimulation (DBS), cortical stimulation (CS), pelvic floor stimulation, gastric stimulation, and the like. The stimulation may be configured to alleviate a variety of symptoms or conditions such as chronic pain, tremor, Parkinson's disease, epilepsy, urinary or fecal incontinence, sexual dysfunction, obesity, or gastroparesis. The stimulation delivered by IMD 12 may take the form of stimulation pulses or continuous waveforms, and may be characterized by controlled voltage levels or controlled current levels, as well as pulse width and pulse rate in the case of stimulation pulses.

IMD 12 may be implanted in patient 60 at a location minimally noticeable to the patient. Alternatively, the device may be external to patient 60 and coupled to implanted leads via a percutaneous extension. For spinal cord stimulation (SCS), as an example, IMD 12 may be located, for example, in the lower abdomen, lower back, or other location to secure the stimulator. Leads 30 may be tunneled from IMD 12 through tissue to reach the target tissue adjacent to spinal cord 62 for stimulation delivery. At distal portions of leads 30 are one or more electrodes that transfer stimulation energy from the lead to the tissue. The electrodes may be electrode pads on a paddle lead, circular (i.e., ring) electrodes, surrounding the body of leads 30, segmented electrodes arranged at different axial and rotational positions around a lead, conformable electrodes, cuff electrodes, or any other type of electrodes capable of forming unipolar, bipolar or multipolar electrode configurations.

The techniques described in this disclosure, including those attributed to processor 16 or various constituent components, may be implemented, at least in part, in hardware, software, firmware or any combination thereof. For example, various aspects of the techniques may be implemented within one or more processors, e.g., processor 16, including one or more microprocessors, digital signal processors (DSPs), application specific integrated circuits (ASICs), field programmable gate arrays (FPGAs), or any other equivalent integrated or discrete logic circuitry, as well as any combinations of such components, embodied in programmers, such as physician or patient programmers, stimulators, image processing devices or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as random access memory (RAM), read-only memory (ROM), non-volatile random access memory (NVRAM), electrically erasable programmable read-only memory (EEPROM), FLASH memory, magnetic data storage media, optical data storage media, or the like. The instructions may be executed to support one or more aspects of the functionality described in this disclosure.

In general, the techniques described in this disclosure may be applied to medical devices such implantable medical devices configured to deliver neurostimulation or other electrical stimulation therapy via implanted electrode arrays, carried by leads or otherwise, located proximate to the spinal cord, pelvic nerves, peripheral nerves, the stomach or other gastrointestinal organs, or within the brain of a patient. The techniques described in this disclosure can be applied to medical devices that may not include electrodes to provide electrical stimulation. For examples, the techniques described in this disclosure can be applied to medical devices that provide medication in accordance with a delivery schedule. The techniques described in this disclosure may also be applied to medical devices that are external to the patient, as well as medical devices that used to program other medical devices.

Many aspects of the disclosure have been described. Various modifications may be made without departing from the scope of the claims. These and other aspects are within the scope of the following claims.

1. A method comprising:
sensing, with a sensor of an implantable medical device (IMD), a first signal that includes a communication key;
receiving, with the IMD, the communication key from the sensed first signal;
coding, with the IMD, at least one of a second signal transmitted by the IMD and a second signal received by the IMD with the communication key received from the sensor; and
sensing, with the sensor that sensed the first signal, a patient characteristic.

2. The method of claim 1, wherein coding, with the IMD, comprises at least one of encrypting and decrypting.

3. The method of claim 1, wherein the sensor is located within at least one of the IMD and a lead coupled to the IMD.

4. The method of claim 1, further comprising:
receiving the second signal from an external device,
wherein sensing the first signal comprises sensing an acoustic signal, and wherein receiving the second signal comprises receiving a radio frequency (RF) signal.

5. The method of claim 1, further comprising:
transmitting the second signal to an external device,
wherein sensing the first signal comprises sensing an acoustic signal, and wherein transmitting the second signal comprises transmitting a radio frequency (RF) signal.

6. The method of claim 1, further comprising:
sensing a third signal, prior to sensing the first signal, that includes an authentication key and a wake-up command;
waking up a telemetry module of the IMD in response to sensing the wake-up command; and
ensuring that the IMD is authorized to communicate with an external device associated with the authentication key in response to sensing the authentication key.

7. The method of claim 1, wherein sensing the patient characteristic comprises at least one of sensing a patient position or movement and sensing a pressure within the patient.

8. The method of claim 1, wherein sensing the first signal comprises sensing an acoustic signal that includes a plurality of digital ones represented by a tone at a first acoustic frequency, and a plurality of digital zeros represented by a tone at a second acoustic frequency, and wherein the acoustic signal comprises the communication key.

9. The method of claim 1, wherein sensing the first signal comprises sensing an acoustic signal that includes a carrier wave modulated at a first acoustic frequency that represents a digital one, and at a second acoustic frequency that represents a digital zero, and wherein the acoustic signal comprises the communication key.

10. The method of claim 1, further comprising receiving updates for at least one of an authentication key and a wake-up command.

11. The method of claim 1, wherein the first signal comprises a first acoustic signal, wherein the second signal comprises a radio frequency (RF) signal, the method further comprising:
sensing, with the sensor of the IMD, a third signal that comprises an acoustic signal and includes an authentication key and a wake-up command, wherein the authentication key and the wake-up command each include a plurality of digital ones represented by a first acoustic frequency and a plurality of digital zeros represented by a second acoustic frequency;
comparing the digital ones and digital zeros of the wake-up command to determine whether to wake-up;
comparing the digital ones and digital zeros of the authentication key to determine whether the IMD is authorized to communicate with an external device associated with the authentication key;
when the IMD is determined to be authorized to communicate with the external device, at least one of decrypting the RF signal, received from the external signal, using the communication key received from the first acoustic signal, and encrypting the RF signal using the communication key received from the first acoustic signal prior to transmitting the RF signal to the external device.

12. An implantable medical device (IMD) comprising:
a sensor operable to sense a first signal that includes a communication key and operable to sense a patient characteristic;
a telemetry module; and
a processor operable to receive the communication key from the sensed first signal, and code at least one of a second signal transmitted by the telemetry module and a second signal received by the telemetry module with the communication key received from the sensor.

13. The IMD of claim 12, wherein the processor is operable to encrypt the second signal transmitted by the telemetry module, and decrypt the second signal received by the telemetry module.

14. The IMD of claim 12, wherein the telemetry module is operable to receive the second signal from an external device, and wherein the first signal comprises an acoustic signal, and the second signal comprises a radio frequency (RF) signal.

15. The IMD of claim 12, wherein the telemetry module is operable to transmit the second signal to an external device, and wherein the first signal comprises an acoustic signal, and the second signal comprises a radio frequency (RF) signal.

16. The IMD of claim 12, wherein the sensor is operable to sense a third signal, prior to sensing the first signal, that includes at least one of an authentication key and a wake-up command, wherein the processor is operable to wake-up the telemetry module in response to the sensor sensing the wake-up command, and ensure that the IMD is authorized to communicate with an external device associated with the authentication key in response to the sensor sensing the authentication key.

17. The IMD of claim 12, wherein the sensor is operable to sense at least one of patient position or movement and a pressure within the patient to sense the patient characteristic.

18. The IMD of claim 12, wherein the first signal comprises an acoustic signal that includes a plurality of digital ones represented by a tone at a first acoustic frequency, and a plurality of digital zeros represented by a tone at a second acoustic frequency, and wherein the acoustic signal comprises the communication key.

19. The IMD of claim 12, wherein the first signal comprises an acoustic signal that includes a carrier wave modulated at a first acoustic frequency that represents a digital one, and at a second acoustic frequency that represents a digital zero, and wherein the acoustic signal comprises the communication key.

20. The IMD of claim 12, wherein the telemetry module is operable to receive updates for at least one of an authentication key and a wake-up command.

21. The IMD of claim 12,
wherein the first signal comprises a first acoustic signal,
wherein the second signal comprises a radio frequency (RF) signal,
wherein the sensor is operable to sense a third signal that comprises an acoustic signal and includes an authentication key and a wake-up command,
wherein the authentication key and the wake-up command each include a plurality of digital ones represented by a first acoustic frequency and a plurality of digital zeros represented by a second acoustic frequency,
wherein the processor is operable to compare the digital ones and digital zeros of the wake-up command to determine whether to wake-up, and is operable to compare the digital ones and digital zeros of the authentication key to determine whether the IMD is authorized to communicate with an external device associated with the authentication key,
wherein when the IMD is determined to be authorized to communicate with the external device, the processor is operable to at least one of decrypt the RF signal, received from the external device, using the communication key received from the first acoustic signal, and encrypt the RF signal using the communication key received from the first acoustic signal prior to the telemetry module transmitting the RF signal to the external device.

22. A system comprising:
at least one external device that transmits a first signal that includes a communication key;
at least one lead that includes a sensor operable to sense the first signal that includes the communication key and operable to sense a patient characteristic; and
an implantable medical device (IMD) implanted within a patient and coupled to the at least one lead, the IMD comprising:
a telemetry module; and
a processor operable to receive the communication key from the sensed first signal, and code at least one of a second signal transmitted by the telemetry module and a second signal received by the telemetry module with the communication key received from the sensor.

23. The system of claim 22, wherein the processor is operable to encrypt the second signal transmitted by the telemetry module, and decrpyt the second signal received by the telemetry module.

24. The system of claim 22, wherein the telemetry module is operable to receive the second signal from the external device, and wherein the first signal comprises an acoustic signal, and the second signal comprises a radio frequency (RF) signal.

25. The system of claim 22, wherein the telemetry module is operable to transmit the second signal to the external device, and wherein the first signal comprises an acoustic signal, and the second signal comprises a radio frequency (RF) signal.

26. The system of claim 22, wherein the at least one external device comprises a first external device, the system further comprising:
a second external device,
wherein the telemetry module is operable to at least one of receive the second signal from the second external device and transmit the second signal to the second external device.

27. The system of claim 22, wherein the sensor is operable to sense a third signal, prior to sensing the first signal, that includes at least one of an authentication key and a wake-up command, wherein the processor is operable to wake-up the telemetry module in response to the sensor sensing the wake-up command, and ensure that the IMD is authorized to communicate with an external device associated with the authentication key in response to the sensor sensing the authentication key.

28. The system of claim 22, wherein the sensor is operable to sense at least one of patient position or movement and a pressure within the patient to sense the patient characteristic.

29. The system of claim 22, wherein the first signal comprises an acoustic signal that includes a plurality of digital ones represented by a tone at a first acoustic frequency, and a plurality of digital zeros represented by a tone at a second acoustic frequency, and wherein the acoustic signal comprises the communication key.

30. The system of claim 22, wherein the first signal comprises an acoustic signal that includes a carrier wave modulated at a first acoustic frequency that represents a digital one, and at a second acoustic frequency that represents a digital zero, and wherein the acoustic signal comprises the communication key.

31. The system of claim 22, wherein the telemetry module is operable to receive updates for at least one of an authentication key and a wake-up command.

32. The system of claim 22,
wherein the first signal comprises a first acoustic signal,
wherein the second signal comprises a radio frequency (RF) signal,
wherein the sensor is operable to sense a third signal that comprises an acoustic signal and includes an authentication key and a wake-up command,
wherein the authentication key and the wake-up command each include a plurality of digital ones represented by a first acoustic frequency and a plurality of digital zeros represented by a second acoustic frequency,
wherein the processor is operable to compare the digital ones and digital zeros of the wake-up command to determine whether to wake-up, and is operable to compare the digital ones and digital zeros of the authentication key to determine whether the IMD is authorized to communicate with the external device,
wherein when the IMD is determined to be authorized to communicate with the external device, the processor is operable to at least one of decrypt the RF signal, received from the external device, using the communication key received from the first acoustic signal, and encrypt the RF signal using the communication key received from the first acoustic signal prior to the telemetry module transmitting the RF signal to the external device.

33. An implantable medical device (IMD) comprising:
means for sensing a first signal that includes a communication key and a patient characteristic;
means for receiving the communication key from the sensed first signal; and
means for coding at least one of a second signal transmitted by the IMD and a second signal received by the IMD with the communication key received from the means for sensing.

34. A non-transitory computer-readable storage medium comprising instructions that, in response to a sensor of an implantable medical device (IMD), which is operable to sense a patient characteristic, sensing a first signal that includes a communication key, cause one or more processors to:
receive the communication key from the sensed first signal; and
code at least one of a second signal transmitted by the IMD and a second signal received by the IMD with the communication key received from the sensor.
